(19) United States
(12) Patent Application Publication
(10) Pub. No.: US 2006/0106334 A1
(43) Pub. Date: May 18, 2006

(54) Breast milk expression system and method

(76) Inventors: Alan E. Jordan, Bend, OR (US); Stephen C. Beal, San Diego, CA (US); Christine M. Kurjan, Mountain View, CA (US); Theo Mann, Palo Alto, CA (US); Bryan T. White, Fremont, CA (US); Gretchen K. Barnes, San Francisco, CA (US); Jennifer E. Davis, Belmont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/815,007

(22) Filed: Mar. 31, 2004

Related U.S. Application Data
(63) Continuation of application No. PCT/US02/31307, filed on Sep. 30, 2002.
(60) Provisional application No. 60/326,755, filed on Oct. 2, 2001.
(60) Provisional application No. 60/326,756, filed on Oct. 2, 2001.
(60) Provisional application No. 60/326,757, filed on Oct. 2, 2001.
(60) Provisional application No. 60/326,751, filed on Oct. 2, 2001.

(51) Int. Cl. A61M 1/06

(52) U.S. Cl. 604/74

(57) ABSTRACT

A system and for discreetly expressing milk from a woman's breast and storing the milk in a container to the side or underneath the breast. The system contains an expression mechanism including various mechanisms for massaging the breast, thereby stimulating the breast to aid in lactation. Both of the user's hands are free during operation of the system allowing the user to perform other tasks during expression or pumping.

### BACKGROUND OF THE INVENTION

1. Field of the Invention

The invention relates to the field of human lactation systems, and more particularly to an improved hands-free breast milk expression system with a breast stimulation mechanism.

2. Description of the Related Art

In recent years the number of women who breast-feed their infants has increased steadily. Breast-feeding is considered to be the best infant feeding option for both mother and child from a nutrition and a psychological point of view. The usual way in which a woman breast-feeds an infant is to let the infant suckle directly from the breast. However, there are a number of circumstances in which it is not convenient or possible for a woman to allow the infant to suckle straight from the breast. Examples of such situations are when the mother is at work and cannot bring the infant with her, when the infant is premature and unable to suckle, or when the mother cannot give milk to the infant for a period of time due to treatment that makes breast-feeding unadvisable. Additionally, for practical reasons, it may also be desirable to enable other persons, such as the father, to feed the infant.

In order to provide breast milk to a child in circumstances where direct breast-feeding is not possible, the alternative of breast milk expression is often used. Breast pump systems for breast milk expression are well known. They use either electric or mechanical means for creating a vacuum to aid the expression of the milk. These systems conventionally have funnel portions that are held against a woman's breast to both introduce the vacuum to the nipples and to catch the expressed breast milk. The vacuum draws breast milk from a nipple through the funnel portion to a breast milk container for subsequent use.

There are a number of physiological and psychological factors which affect milk production. An essential factor is the stimulation of the breast to initiate milk production. For example, stimulation of the breast, especially at the edge of the areola and the nipple, causes the release of the hormone prolactin. This hormone has a direct impact on the lactiferous ducts so that these are expanded and opened for depletion. This happens when the infant grasps the breast in its mouth. The baby will then stimulate the areola and the nipple with its lips and tongue.

A number of different funnels or breast cups have been developed, which to some degree, stimulate the area around the nipple. However, stimulation of only this area is not sufficient to instigate satisfactory milk production. A major disadvantage with these funnels is they do not provide adequate stimulation to areas of the breast not in the area immediately around the nipple. A second disadvantage experienced with these funnels is that they are made of inflexible materials that are not sufficiently capable of adapting to the shape of the breast. This can cause the user some discomfort. In addition to being capable of stimulating the breast, the breast pump should also be comfortable to use and have a breast cup that is soft and adaptable to different shapes and sizes of breasts. At the same time, the breast pump must be easy to use.

Typically, the expression of milk requires the use of the mother's hands to manipulate the pump and or receptacle. Thus, full attention must be given to the process. To address this problem, hands-free breast pump support devices have also been developed. However, these devices have many different parts that make their use very complicated, difficult and time-consuming. Accordingly, they are essentially ineffective in achieving true hands-free operation. Also, many of these hands free devices simply hold a conventional breast pump and collection bottle on the breast in a cumbersome fashion. The devices do not allow the mother to discreetly express milk while performing other tasks. In addition, the use of these devices is often so complicated that many women refuse to use them.

Therefore, it would be desirable to have an easy-to-use, hands-free breast milk expression system with an attached collection container that allows a woman to discreetly express breast milk. It is desirable to have a system that simultaneously supports an expression mechanism and funnel portion of a breast milk expression device against each breast. It is desirable to have a breast milk expression system that is a time-saving device for nursing mothers which allows the nursing mother to wear a device that leaves her hands free for tending to the child or doing other things.

### Summary of the Invention

The systems and methods of the invention have several features, no single one of which is solely responsible for its desirable attributes. Without limiting the scope as expressed by the claims that follow, its more prominent features will now be discussed briefly. After considering this discussion, and particularly after reading the section entitled “Detailed Description of the Preferred Embodiments” one will understand how the features of the system and methods provide several advantages over traditional systems and methods.

In one aspect of the invention, a system is described for expressing breast milk including a flexible breast shield, a cup attaching to the breast shield, a collection container connected to the cup for collecting expressed milk, and an expression mechanism for stimulating the breast. In one embodiment, the collection container attaches to the cup via a flexible duct and includes an outer container and an inner container. In an alternate embodiment, the collection container connects directly to the breast shield and includes a flexible bag that holds the milk underneath the breast.

In another aspect, the expression mechanism includes pressurized chambers. The pressurized chambers are filled with a fluid such as air and are cyclically pressurized in order to stimulate the breast. In another aspect, multiple rows of pressurized chambers are arranged around the breast.

In yet another aspect, the expression mechanism includes a plurality of beads attached to flexible members. An actuator acts upon the flexible member, causing the beads to roll along the breast. In another embodiment, the actuator causes the beads to roll across the breast in a rocking manner.

In still another aspect, the expression mechanism includes a plurality of flexible members. An actuator acts upon the flexible members, causing the contact area of the flexible members to roll along the breast. In another aspect, an electrical current causes the contact area of the flexible members to roll along the breast.

In another aspect, the expression mechanism includes a plurality of helical members attached to transmission ring. An actuator acts upon the helical members, causing the helical members to rotate, thereby applying pressure to the breast. In another aspect, the expression mechanism includes a contracting ring with compression and tension members that cause the ring to expand and contract.

### BRIEF DESCRIPTION OF THE DRAWINGS

These and other objects and features of the invention will become more fully apparent from the following description and appended claims taken in conjunction with the following drawings, where like reference numbers indicate identical or functionally similar elements.

FIG. 1 is a block diagram of an embodiment of a breast milk expression system according to the invention.

FIG. 2 is a perspective view of one embodiment of a milk collection system of the expression system of FIG. 1.

FIG. 3 is a side cross sectional view of a cup used in the collection system of FIG. 2 and taken along line 3-3 of FIG. 2.

FIG. 4 is a cross sectional view of another embodiment of a milk collection system of the expression system of FIG. 1 taken along line 4-4 of FIG. 2.

FIG. 5 is a perspective view of another embodiment of a milk collection system of the expression system of FIG. 1

FIG. 6 is a perspective view of one embodiment of a collection container used with the milk collection system of FIG. 5.

FIG. 7 is a perspective view of one embodiment of a collection container, illustrating its use in the collection system of FIG. 2.

FIG. 8 is a perspective view of an embodiment of an expression mechanism of the expression system of FIG. 1 having pressurized chambers.

FIG. 9 is a cross-sectional side view of the pressurized chambers of the expression mechanism of FIG. 8 taken along line 9-9 of FIG. 8.

FIG. 10 is a perspective view of another embodiment of an expression mechanism of the expression system of FIG. 1 having rows of pressurized chambers.

FIG. 11 is a cross-sectional side view of the rows of pressurized chambers of the expression mechanism of FIG. 10 taken along line 11-11 of FIG. 10.

FIG. 12 is a perspective view of another embodiment of an expression mechanism of the expression system of FIG. 1 having discrete pressurized points.

FIG. 13 is a perspective view of an embodiment of an expression mechanism of the expression system of FIG. 1 having an array of beads arranged on flexible members.

FIG. 14 is a side view of the flexible members of FIG. 13 attached to an actuator.

FIG. 15 is a perspective view of another embodiment of an expression mechanism of the expression system of FIG. 1 having an array of beads arranged on a flexible finger.

FIG. 16 is a side view of the flexible finger of FIG. 15 attached to a motor.

FIG. 17 is a perspective view of another embodiment of an expression mechanism of the expression system of FIG. 1 having peg-like members arranged on strings.

FIG. 18 is a side view of the strings of FIG. 17 attached to an actuator.

FIG. 19 is a perspective view of another embodiment of an expression mechanism of the expression system of FIG. 1 having beads arranged on a cable.

FIG. 20 is a perspective view of an embodiment of an expression mechanism of the expression system of FIG. 1 having multiple helical members.

FIG. 20a is a front view of the transmission ring, motor and rotary input of the mechanism of FIG. 20.

FIG. 21 is a perspective view of another embodiment of an expression mechanism of the expression system of FIG. 1 having an expanding and contracting ring.

FIG. 22 is a cross-sectional side view of the ring of FIG. 21 with compression and tensioning members taken along line 22-22 of FIG. 21.

FIG. 23 is a perspective view of another embodiment of an expression mechanism of the expression system of FIG. 1 having an expanding and contracting spiral.

FIG. 23a is a front view of the spiral winding of the expression mechanism, isolated from the rest of the system.

FIG. 24 is a perspective view of an embodiment of an expression mechanism of the expression system of FIG. 1 having flexible fingers.

FIG. 25 is a cross-sectional side view of the flexible fingers of the expression mechanism of FIG. 24 taken along line 25-25 of FIG. 24.

FIG. 25a is a side view of one of the flexible fingers of the expression mechanism of FIG. 25 that demonstrates the movement of the flexible finger under the influence of electric current.

FIG. 25b is a side view of one of the flexible fingers of the expression mechanism of FIG. 25 that demonstrates the movement of the flexible finger under the influence of pressurization.

FIG. 26 is a perspective view of an embodiment of an expression mechanism of the expression system of FIG. 1 having discrete pressure points.

FIG. 27 is a perspective view of another embodiment of the expression system of FIG. 1 in which the expression system can be repositioned in a conventional bra.

FIG. 28 is a block diagram showing the method of using the expression system of FIG. 1.

### DETAILED DESCRIPTION OF THE INVENTION

Embodiments of the invention will now be described with reference to the accompanying figures, wherein like numerals refer to like elements throughout. The terminology used in the description presented herein is not intended to be interpreted in any limited or restrictive manner simply because it is being utilized in conjunction with a detailed description of certain specific embodiments of the invention. Furthermore, embodiments of the invention may include several novel features, no single one of which is solely responsible for its desirable attributes or which is essential to practicing the inventions herein described.

The invention deals generally with a new and improved system for expressing milk from the breasts of a nursing mother. In one embodiment, the apparatus allows the nursing mother to simultaneously express milk from the right and left breast in a discrete manner while leaving both of the nursing mother's hands free to perform other tasks. However, the system can be used to pump from one breast or the other at any one time to exclusion of the other breast as well.

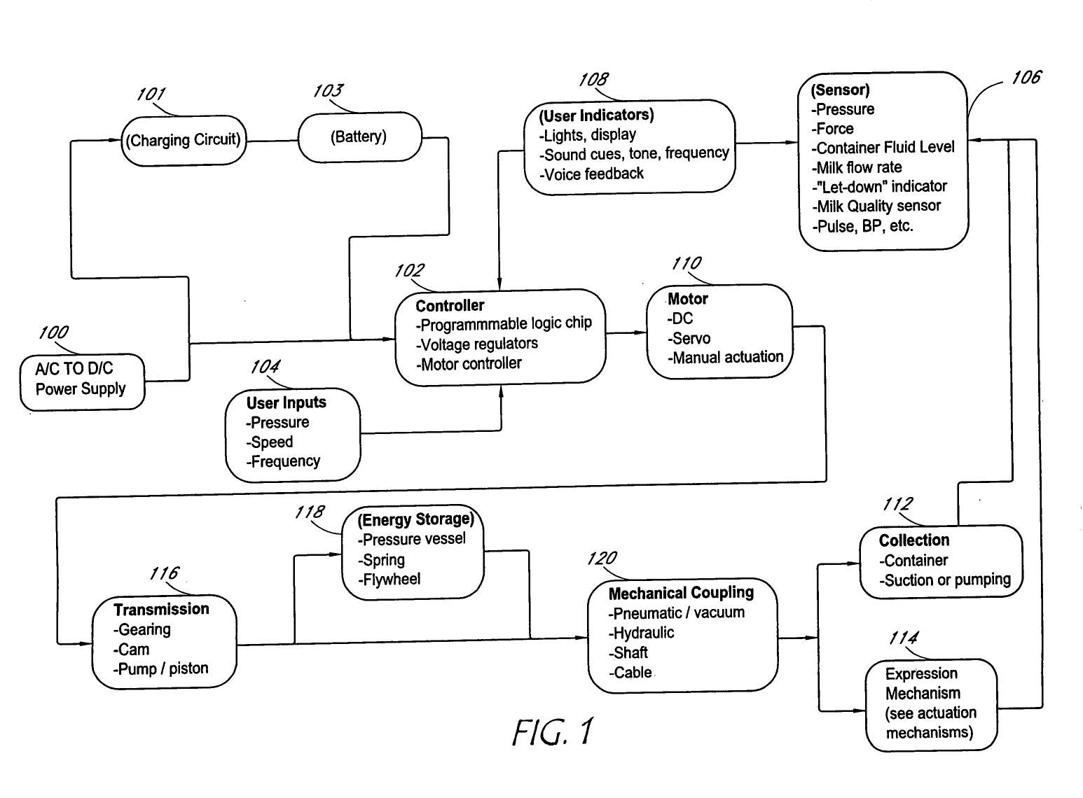

FIG. 1 is a high-level block diagram illustrating components included in a breast milk expression system. The system includes a power supply 100 connected to a controller 102. Various types of power supplies 100 are contemplated, such as A/C, an A/C to D/C converter, or a battery charging circuit 101 and battery 103. The controller 102 allows the user to enter user inputs 104 to control such parameters as pressure, speed or frequency of components of the system, as will be described below. The controller 102 receives inputs from sensors 106 that determine various parameters such as pressure, milk flow rate, container fluid level, milk quality, or vital statistics of the mother, such as pulse rate or blood pressure. The controller 102 also displays various user indications 108 with lights, sounds or voice feedback, allowing the user to determine the settings of the controller 102 and receive the indications from the various sensors. The controller 102 controls a motor 110, such as a servomotor or a DC motor, for operation of components in the system as will be described below. The controller 102 can have a programmable logic chip to control the user inputs and coordinate the sensors and user indications.

As shown, the system also includes a milk collection system 112 and an expression mechanism 114, both of which will be described in detail below. The motor 110 may connect to the collection system 112 and expression mechanism 114 through a transmission device 116, an energy storage device 118, and a mechanical coupling 120. The controller 102 provides the user with manual control of the expression mechanism and/or automatic control of expression mechanism, through the use of a timer and/or flow control sensors that sense when milk is no longer being expressed. Each of the individual components is specifically configured and correlated one with respect to the other so as to attain the desired objectives.

Milk Collection System

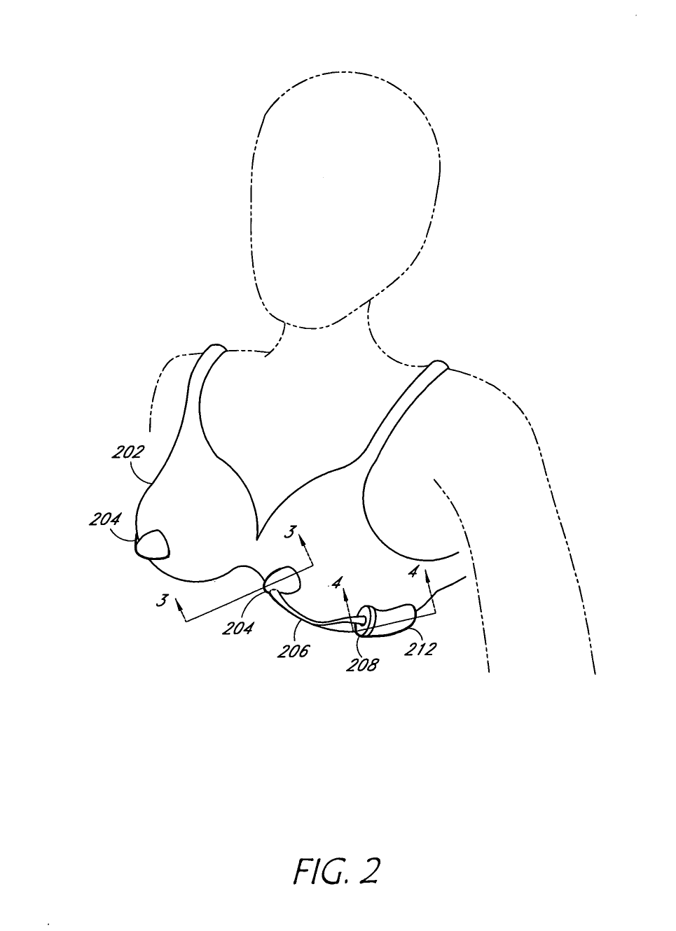

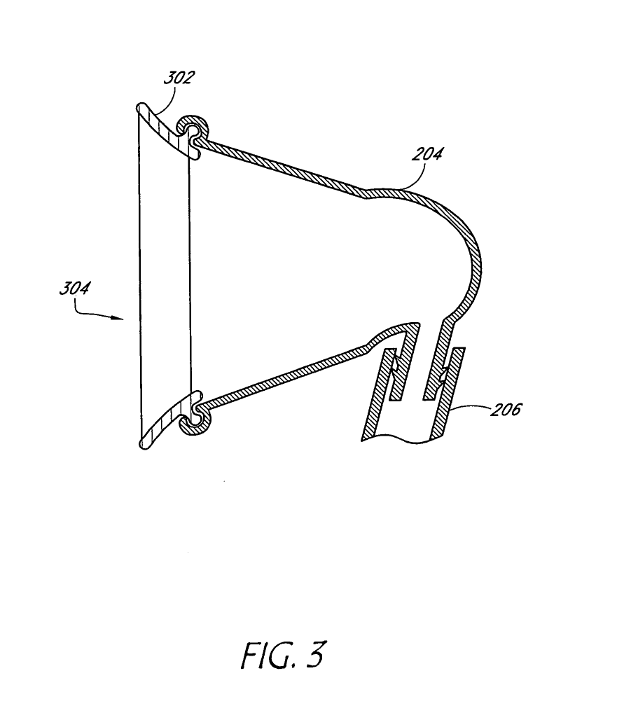

Referring now to FIGS. 2 and 3, one embodiment of a milk collection system 112 is shown. The expression mechanism 114, controller 102 and motor 110 have been removed from these figures for clarity. FIG. 2 illustrates a bra 202, or other vest-like article of clothing, that is worn by the nursing mother. The bra can be made of a conventional material and may include a heating mechanism (not shown) and a cooling mechanism (not shown) to aid in stimulating the breast. The heating mechanism can use conventional flexible thermal circuits.

Referring to FIG. 3, contact with the breast of the mother is made through a pair of nipple shields 302 on the interior surface of each of the cups of the bra 202. Each of the shields 302 is formed from a generally cone shaped configuration and is positionable over the breast of the nursing mother. The breast shield 302 contacts the breast to provide a seal that prevents milk from escaping the system and allows the application of suction by preventing excessive influx of ambient air. The seal can be formed by creating a suction pressure in the shield, use of a gel seal, or with adhesives. The shield 302 can be made of a flexible plastic or glycerin based product that conforms to the shape of the breast. A suction drive unit (not shown) can generate the suction pressure through duct 206 or other suitable connection.

Each of the shields 302 is also formed to have a centrally located aperture 304. A nipple cup 204 is attached over the aperture 304 that surrounds the nipple and collects the milk produced in lactation. In the embodiment illustrated in FIG. 3, the nipple cup 204 snaps on to the outside of the aperture 304, but any attachment mechanism can be used. Each cup 204 is attached to a transport tube or duct 206. As shown, the cup may have ridges where it contacts the duct 206 to maintain the connection with the duct 206, however, a clip can be used as well as any other mechanism for maintaining the Referring again to FIG. 2, it is seen that the duct 206 connected to the cup 204 over the right breast is connected to a fitting or cap 208 positioned on the right side of the mother's body or breast. The duct 206 connected to the cup 204 over the left breast is attached to a cap 208 positioned on the left side of the mother's body or breast. The ducts 206 can be removably attached to the cups 204 and to the cap 208 to aid in clean up after use. Each cap 208 is attached to a collection container 212 by conventional means such as a screw fitting, clamp, or adhesive that allows the collection container 212 to be removed by the user but prevents the spillage or escape of milk from the system when attached to cap 208. Other means of securing the cap 208 to the collection device as are commonly known may also be used. Milk is thus stored in the container 212, which is positioned on the body to the sides of the breasts, having been collected at cups 204 and transported through ducts 206 and caps 208.

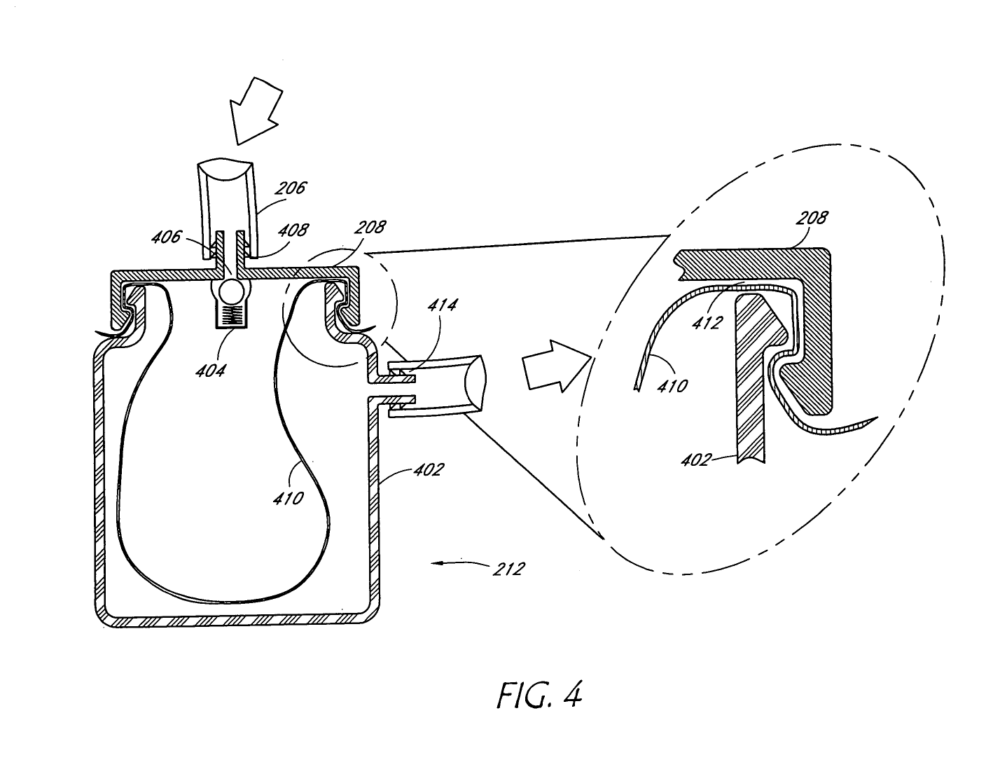

FIG. 4 shows a cross sectional view of an embodiment of the collection container 212. The collection container 212 includes an outer container 402 that connects to the cap 208. A one-way valve 404 is placed in an opening 406 of the cap 208 to substantially prevent the backflow of milk from the collection container 212 back to the cup 204. The duct 206 attaches to a nozzle 408 on the cap 208. In one embodiment, the duct 206 can attach by a frictional fit to the nozzle 408. Alternately, the nozzle 408 and duct 206 may be provided with threaded portions and the duct can be screwed onto the cap 208. The one-way valve 404 is preferably made from a material that is flexible, durable, non-toxic and compatible with milk. The one-way valve 404 can be, for example, a flapper valve, a poppet, or a tri-cuspid; however, any type of one-way valve that provides smooth passage of milk from the cup 204 into the collection container 212 and discourages the return of milk through the valve 404 may be used. The one-way valve 404 may alternately be incorporated as part of the duct 206.

The outer container 402 preferably is made of a rigid or semi-rigid material, of a type that will not easily collapse if it is squeezed or smashed. An inner container 410 is placed inside the outer container 402 such that the inner container is held in place with an opening facing the duct 206. As shown, a rim 412 of the inner container 410 is held by frictional fit between the outer container 402 and the lid 208. The outer container 402 also has a second suction duct 414 in a wall thereof. Through the suction duct 414, a vacuum is drawn in the outer container 402 causing the inner container 410 to conform to the inner walls of the outer container 402 and assist in the collection of milk into the inner container 410. The inner container 410 can be a plastic, non-toxic bag suitable for holding milk, which can be transferred directly into a baby bottle.

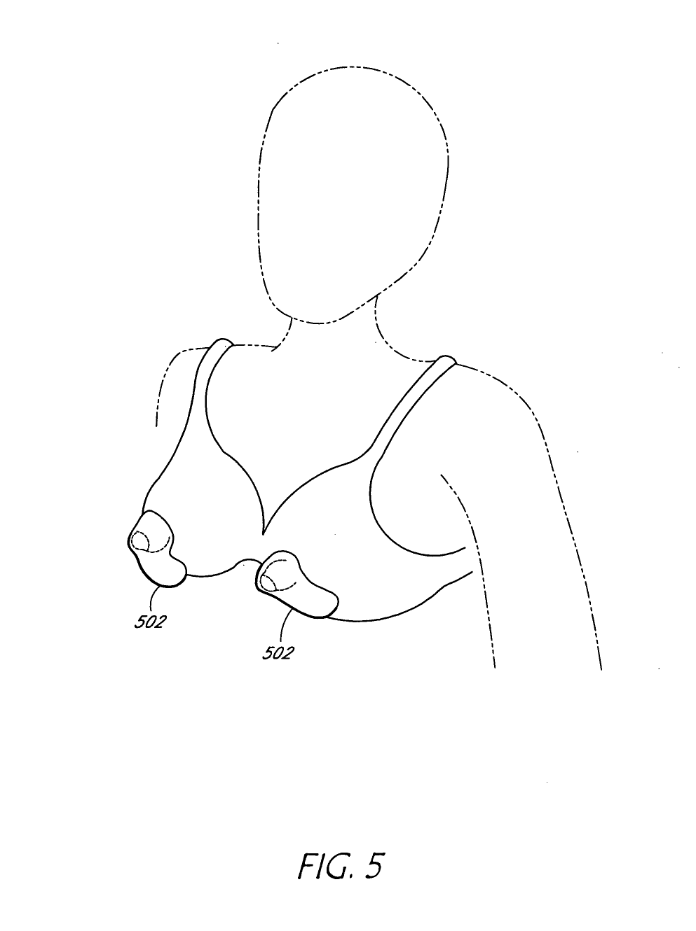

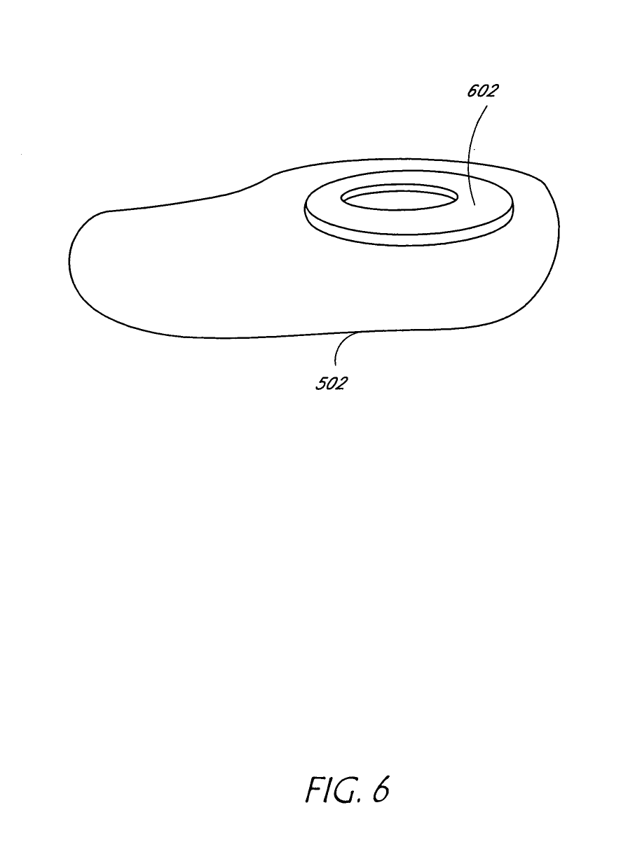

FIG. 5 illustrates another embodiment of a collection system 112. This system similarly attaches to the breast with a breast shield 302 (not shown) that is connected directly to a collection container 502. The collection container 502 can be a flexible bag made of plastic or other material suitable for storing milk. The collection container 502 stores the milk next to the body and under the breasts. FIG. 6 illustrates that the collection container 502 has a rim 602 that can attach to the shield 302 to prevent leakage between the shield 302 and the collection container 502.

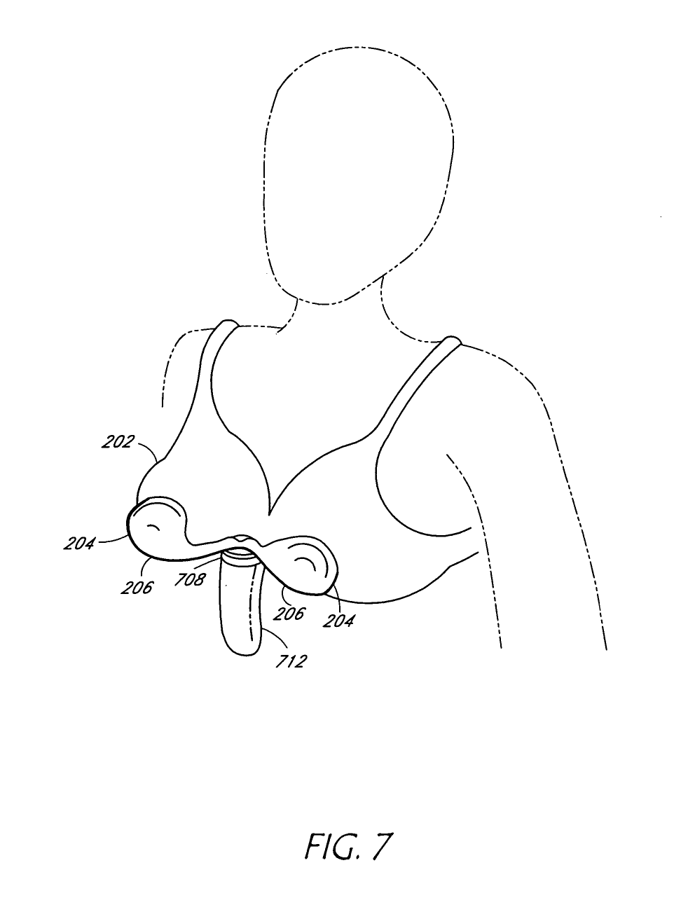

FIG. 7 illustrates another embodiment of a collection container 112. This system similarly attaches to the breasts with a breast shield 302 that connects to a nipple cup 204. As shown, ducts 206 attach to each cup 204 in a similar manner as explained above. However, the ducts 206 connect to a single cap 708. The cap 708 attaches to a single collection container 712 that stores the milk next to the body between and below the breasts. The collection container 712 can have an inner container and an outer container as explained above.

Expression Mechanism

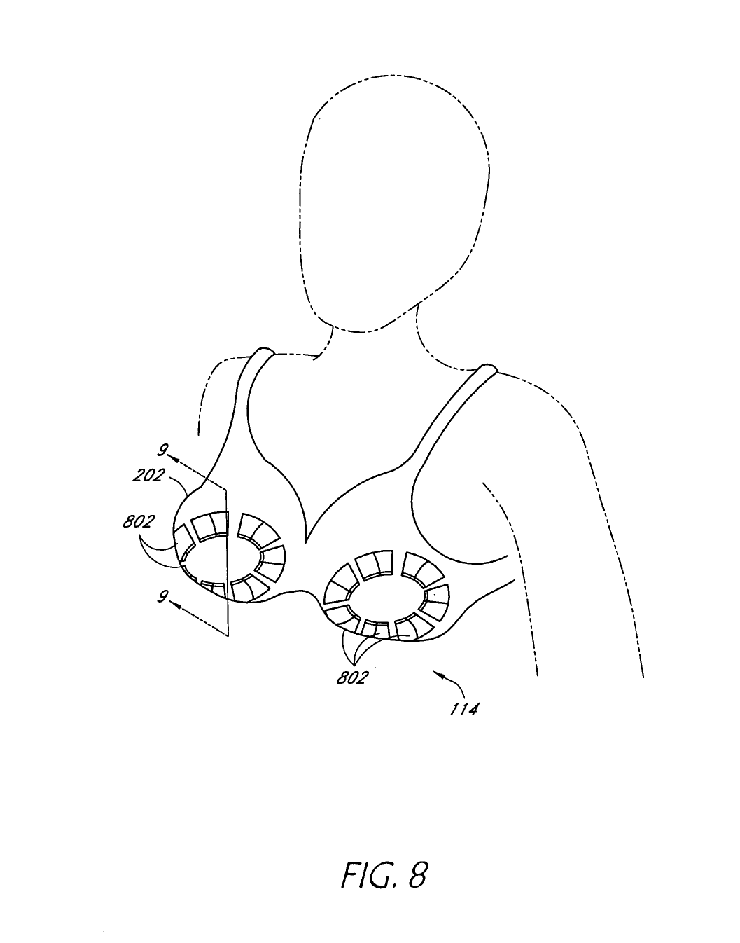

FIG. 8 illustrates an expression mechanism 114 attached to the bra 202 with the collection system 112 removed for clarity. The expression mechanism acts upon the breast through a series of pressurized chambers 802 that are held against the breast by the bra 202 in order to express the milk through the nipple. The chambers 802 are supplied with a pressurized fluid, preferably compressed air. The pressurized chambers 802 inflate and press against an area of the breast to stimulate the breast. When pressure is relieved, the chambers 802 contract, thereby no longer pressing on the breast. The chambers 802 are arranged concentrically around the nipple (or in other radiating patterns) and can be cyclically pressurized in order to produce patterns of pressure against the breast to assist in expressing milk. The system may employ a rigid backing (not shown) to the chambers 802 that will counter the force of the chambers and maintain the pressure firmly against the breast.

The chambers 802 are connected to a pressure pump (not shown) that is capable of producing an oscillating pressure in the chambers 802. The user is able to control pressure, frequency and pattern of stimulation with the controller 102 of FIG. 1 to maximize the effect of breast stimulation.

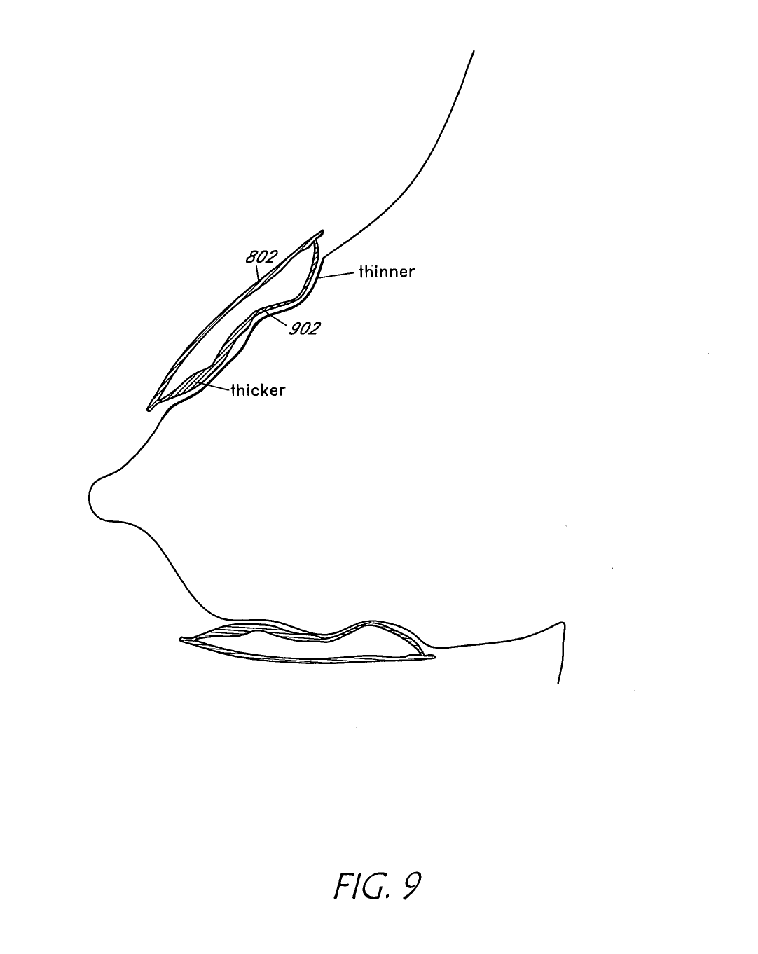

FIG. 9 illustrates that in one embodiment, the interior walls 902 of the chambers 802 have a variable thickness. Preferably, the wall 902 is thinner in the portion of the wall furthest from the nipple and thicker in the portion of the wall nearest the nipple. In this manner, a variable pressure will be felt on the breast producing a pulsating wave from the area furthest from the nipple towards the nipple.

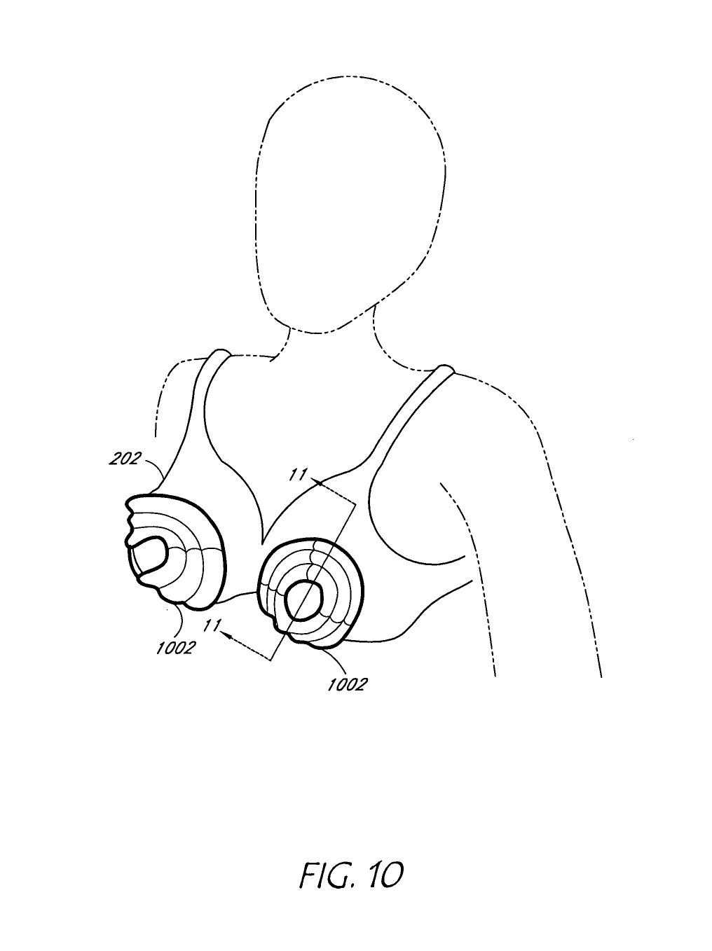

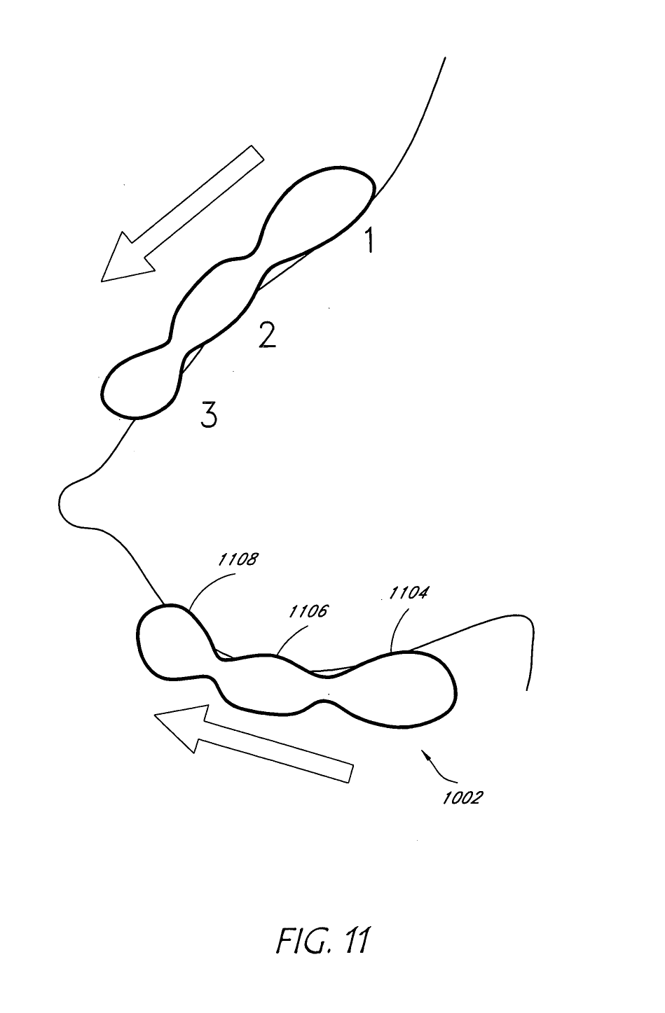

FIG. 10 illustrates another embodiment with multiple rows of pressurized chambers 1002. The chambers 1002 are filled with a pressurized fluid and connected to a pump as described above. FIG. 10 illustrates that the rows of chambers 1002 can be cyclically pressurized in order to produce patterns of pressure against the breast. FIG. 11 illustrates that preferably, an outer row 1104 of the chamber 1002 is pressurized first, then a middle row 1106, and finally, an inner row 1108, to produce a pulsating pressure wave from the area furthest from the nipple towards the nipple.

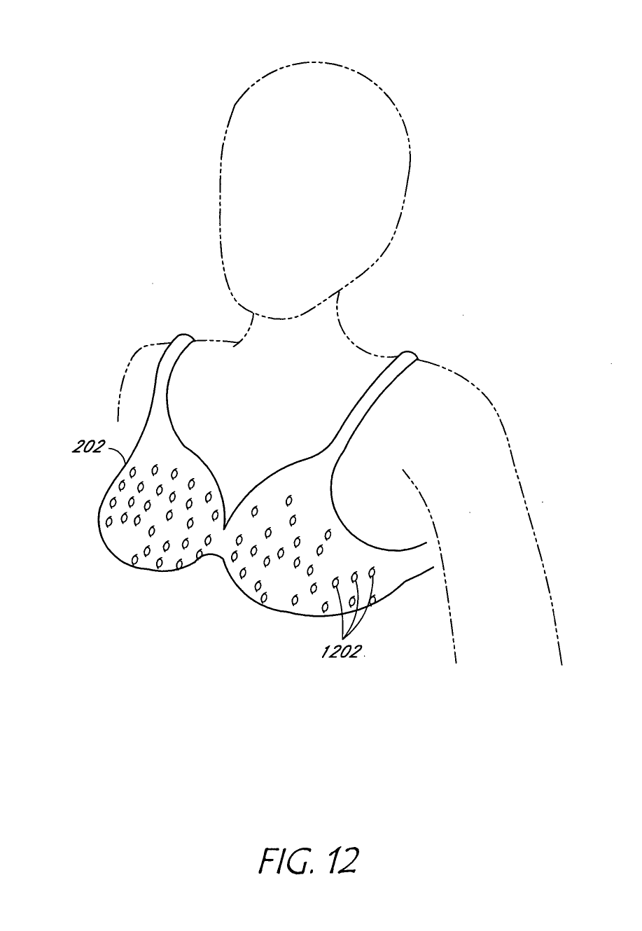

FIG. 12 illustrates another embodiment of an expression system 1214 that uses a series of discrete actuation points 1202 arrayed around the nipple and over the breast to provide a configurable pattern of stimulation. Each actuation point 1202 is supplied with a pressurized fluid. A system for providing each discreet actuation point 1202 with fluid pressure includes any tubing, vales and pumps those of skill in the art would use to create the necessary pressurization of the system. Any such system meeting the functions described herein can be used to fulfill these functions. In some embodiments, such pressurization systems will form part of the bra 202, while in other embodiments, the pressurization systems will be secured to a portion of the bra 202.

In some embodiments this pressurized fluid is compressed air. The actuation points 1202 inflate and press against an area of the breast to stimulate the breast. When pressure is relieved, the actuation points 1202 no longer press on the breast. The actuation points 1202 can be arranged concentrically around the nipple (or in other radiating patterns) and are cyclically pressurized in order to produce patterns of pressure against the breast to assist in expressing milk. The pattern, frequency and pressure exerted by the actuation points 1202 are controlled by the user using the controller 102 (FIG. 1) in order to maximize the effectiveness for milk expression. In such embodiments, the various pressurization sequences may be pre-programmed into the controller by the manufacturer or the user and the controller circuitry (not shown) performs the necessary functions known to those of skill in the art to pressurize and de-pressurize the various discrete actuation points in the appropriate sequence to fulfill the intended functions described herein. Typical patterns will begin pressurizing the actuation points 1202 furthest from the nipple first and then work there way toward the nipple, however any pattern can be used.

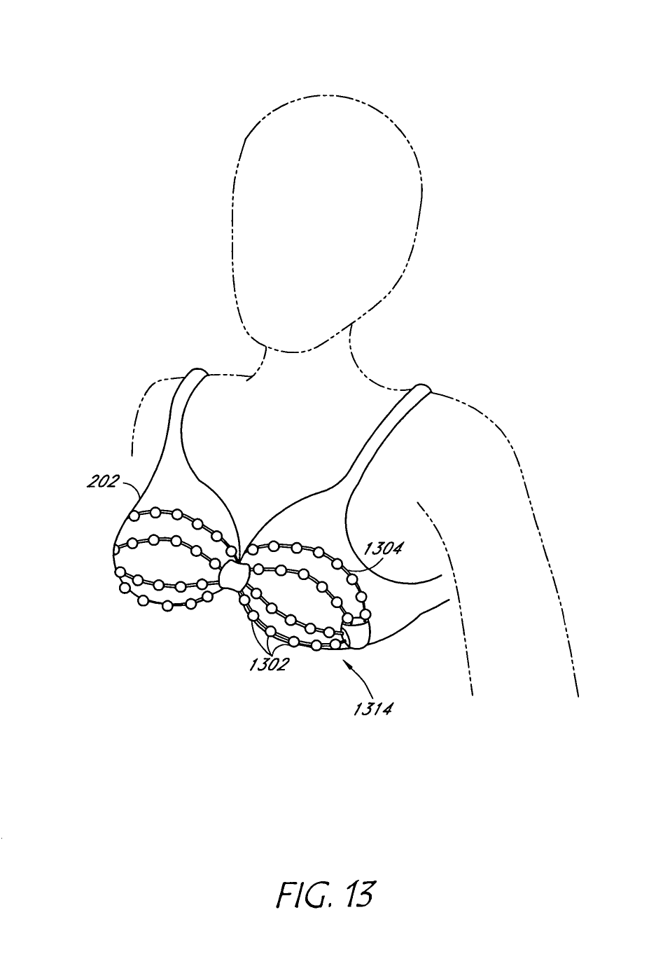

FIG. 13 illustrates an expression mechanism 1314 attached to the bra 202 with the collection system 112 removed for clarity. The expression mechanism 1314 acts upon the breast through an array of balls, rollers, or beads 1302 that are arranged along the length of a several flexible members 1304. The flexible members 1304 form to the contour of the breast and press the beads 1302 against the breast. Multiple flexible members 1304 may be arranged above and below the nipple area.

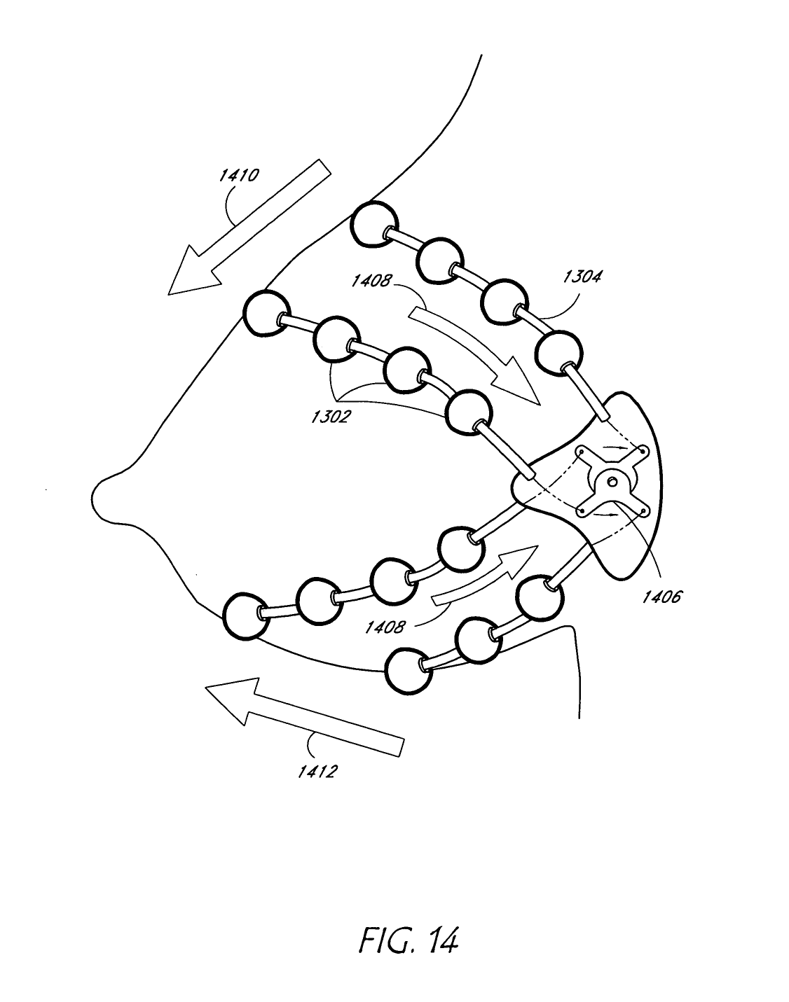

FIG. 14 illustrates that an actuator 1406 is connected to the bra 202 and acts upon flexible members 1304, pulling the flexible members 1304 in a manner such that the beads 1302 move along the breast in a first direction 1408 towards the actuator and also causing the beads 1302 to move in a second direction 1410 towards the nipple. In one embodiment, the beads 1302 roll along the breast. The moving or rolling motion of beads 1302 provides a massaging action that will cause or aid in the expression of milk from the nipple. The motor 106 (see FIG. 1) causes the actuator 1406 to rotate, thereby pulling the flexible members 1304.

In one embodiment, the flexible members 1304 are made of an elastomeric material and the ends of flexible members 1304 opposite to the actuator 1406 are attached to bra 202. When the force is removed from the actuator 1406, the flexible members 1304 contract, returning the beads 1302 to the original position. In one embodiment, the actuator 1406 may be supplemented by a secondary actuator (not shown) attached to the opposite end of the flexible members 1304, which operate in alternate fashion with the actuator 1406 to thereby create a back and forth motion of the beads 1302 across the breast. The user is able to control tension of the flexible members 1304 and the frequency of oscillation with the controller 102 of FIG. 1 to maximize the effect of breast stimulation.

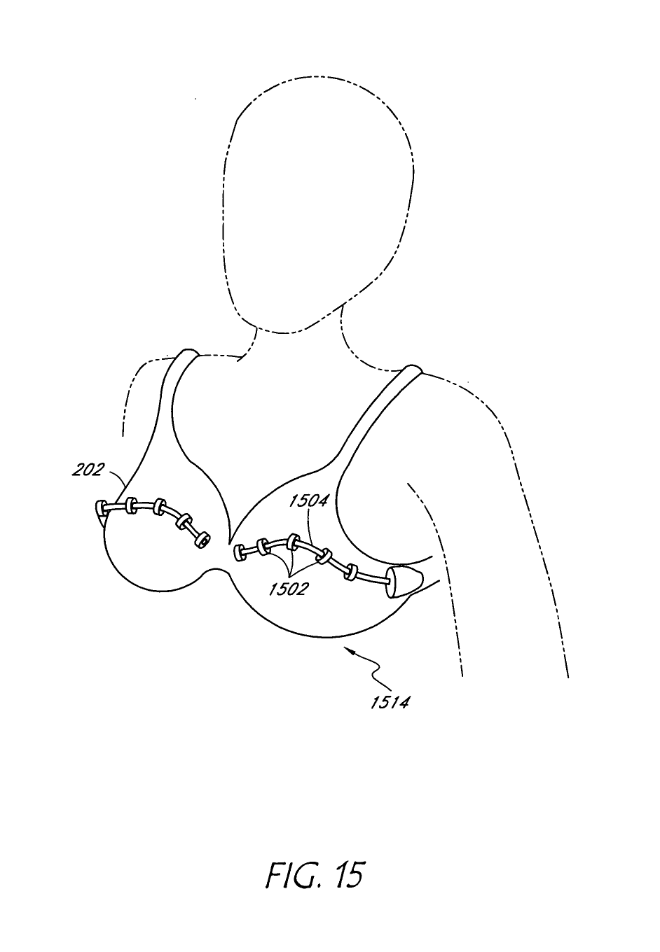

FIG. 15 illustrates another embodiment of an expression mechanism 1514 attached to the bra 202 with the collection system 112 removed for clarity. The expression mechanism 1514 acts upon the breast through an array of balls, rollers, or beads 1502 that are arranged along the length of a finger 1504. The finger 1504 forms to the contour of the breast and presses the beads 1502 against the breast.

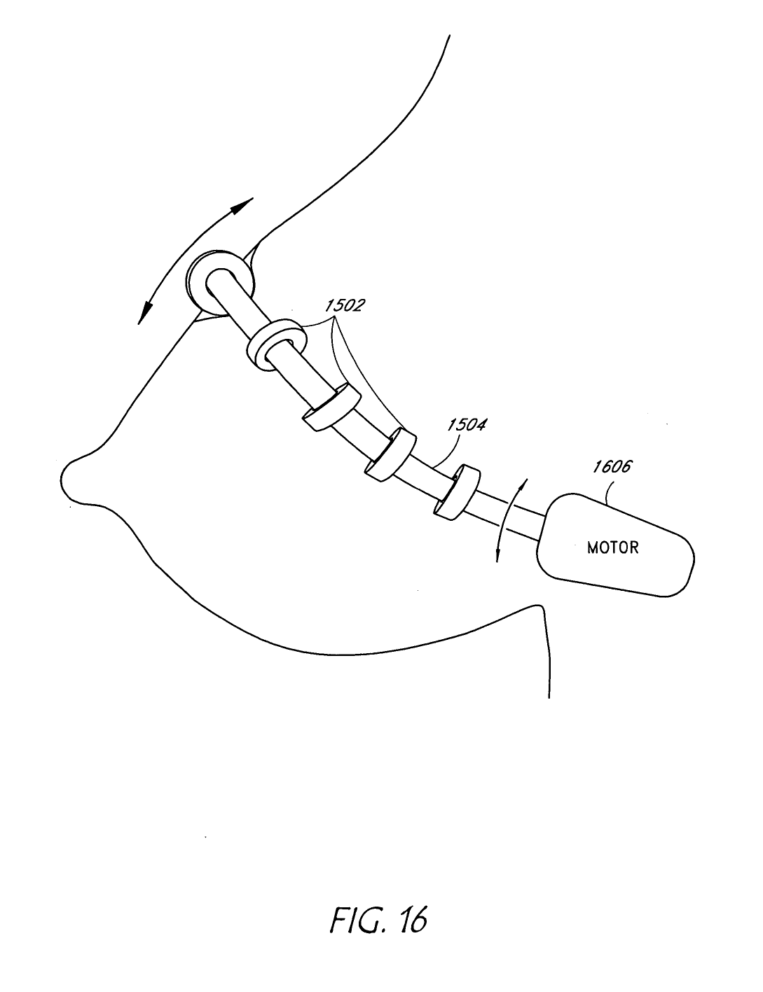

FIG. 16 illustrates that a motor 1606 is secured to the bra and acts upon the finger 1504 in a manner such that the beads 1502 roll along the breast. The rolling motion of beads 1502 provides a massaging action that will cause or aid in the expression of milk from the nipple. The user is able to control the frequency of the motor 1606 with the controller 102 of FIG. 1 to maximize the effect. In some embodiments, the finger 1504 applies greater pressure when stroking towards the nipple and less pressure on the return stroke away from the nipple.

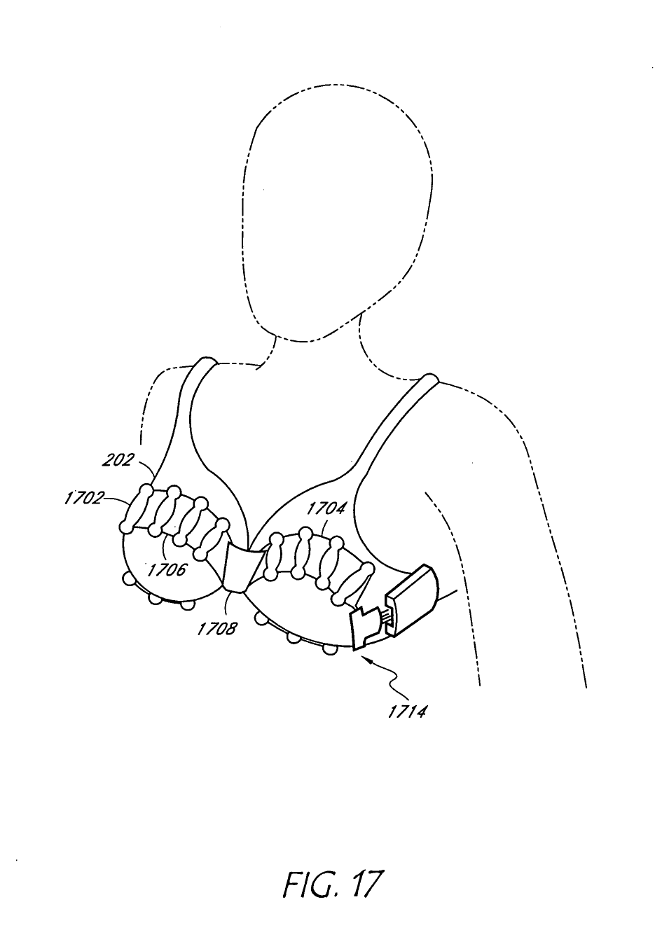

FIG. 17 illustrates another embodiment of an expression mechanism 1714 attached to the bra 202 with the collection system 112 removed for clarity. The expression mechanism 1714 acts upon the breast through peg-like elements 1702 that are arranged around the nipple and radiate away from the nipple. These pegs 1702 can be caused to rock along radians of the breast thus massaging it to cause or aid expression of milk. Preferably, the pegs 1702 are attached to an upper string 1704 and a lower string 1706. The upper string 1704 passes through an outer end of each bead 1702 and the lower string 1706 passes through an inner end of each bead 1702. The strings 1704, 1706 help keep the pegs 1702 close against the breast. The strings 1704, 1706 can be fixed at one end to an attachment location 1708, in this case the front and center of the bra 202.

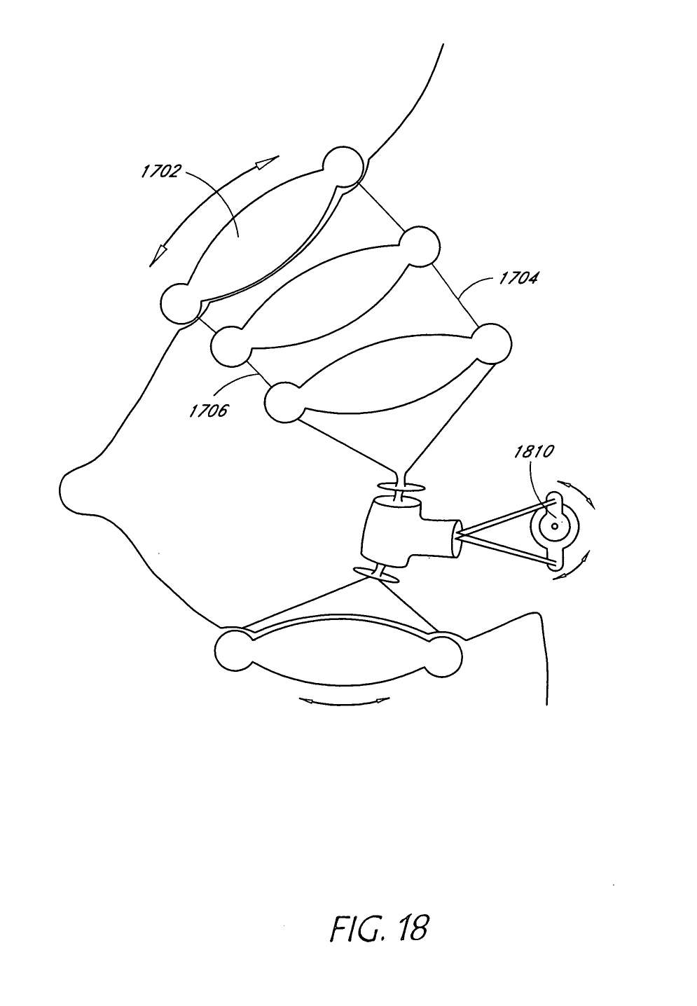

FIG. 18 illustrates that the strings 1704, 1706 are connected to an actuator 1810, which is secured to the bra 202. The actuator 1810 pulls alternately on the upper string 1704 and the lower string 1706, with separate string connections for each (not separately identified), to shorten the exposed length. In the embodiment, illustrated, the strings 1704, 1706 pass through a string director piece (not separately identified) before passing to the upper and lower sides of the mechanism 1714. This director piece can also be secured to the bra 202 or may be suspended by the strings in its position. The alternate pulling of the strings 1704, 1706 causes a contraction of the outer portion of the circle, followed by a release of the outer circle and contraction of the inner circle. The actuator repeats the motion to achieve a rocking motion of the pegs 1702. The user is able to control the frequency of movement of the actuator 1810 with the controller 102 of FIG. 1, similar to embodiments described above. Some embodiments will secure at least some portions of the strings 1704, 1706 to the bra as well to prevent their movement around the surface of the breast. Such securing can be done by any method allowing the expression mechanism 1714 to function as described above.

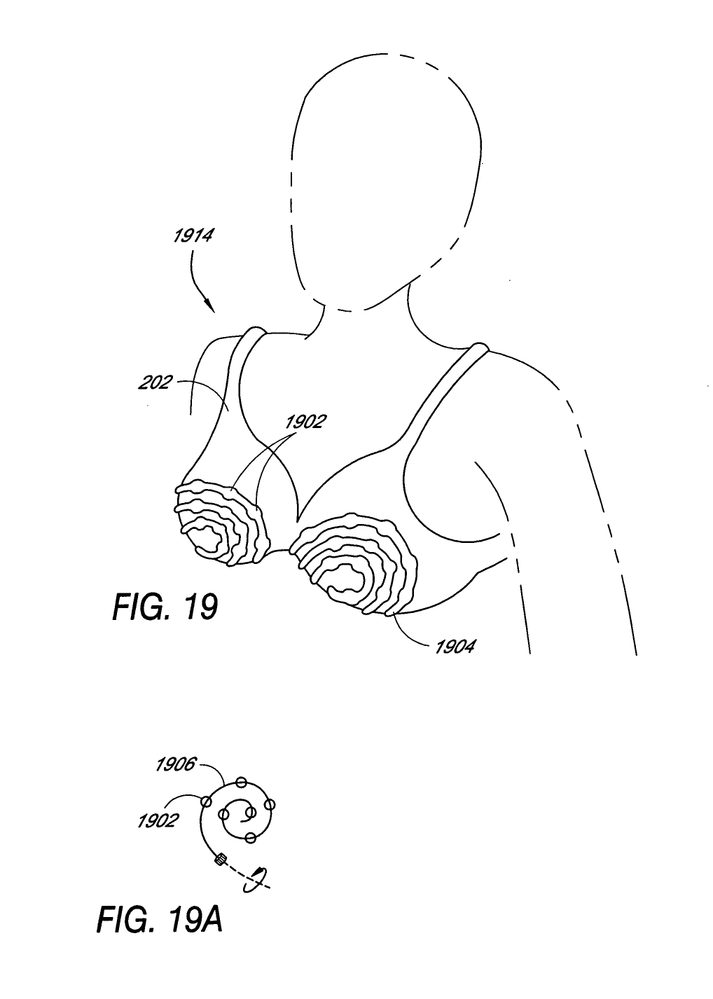

FIG. 19 illustrates another embodiment of an expression mechanism 1914 attached to the bra 202 with the collection system 112 removed for clarity. The expression mechanism 1914 acts upon the breast through a cable 1904 with an array of balls, rollers, or beads 1902 that are arranged along the length of the cable 1904. FIG. 19a illustrates that the cable 1904 has a wire 1906 encased in the flexible cable 1904. The wire 1906 rigidly attaches to the beads 1902. Torque applied at one end of the cable 1904 will produce the same twisting throughout the length of the cable 1904, causing the beads 1902 to rotate. This produces an intermittent pressure at discrete points about the breast.

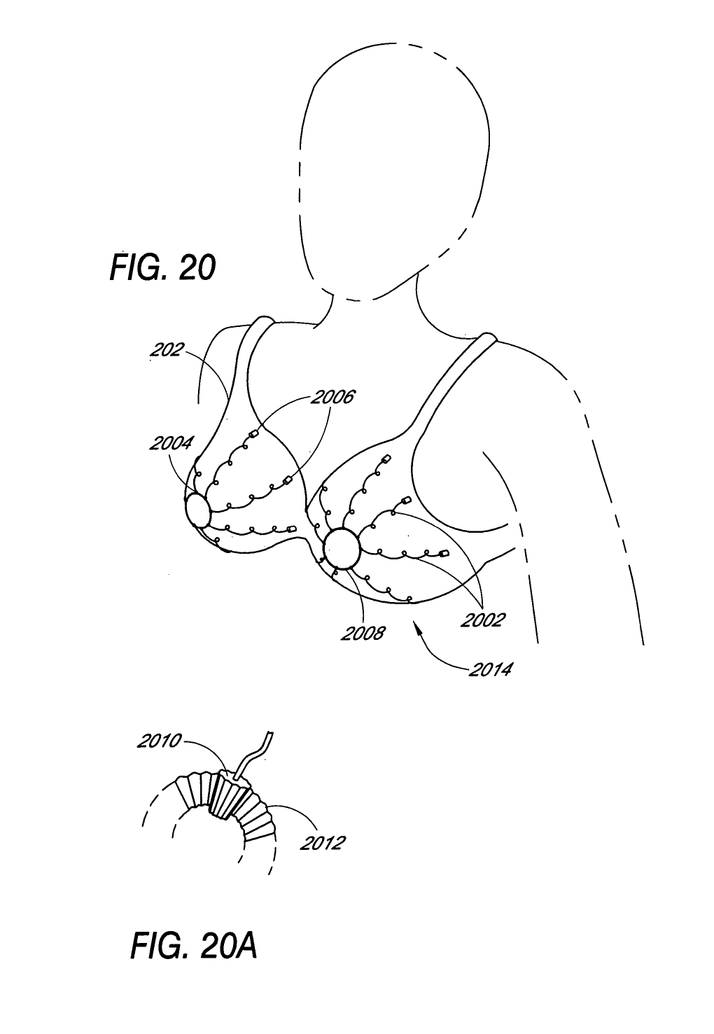

FIG. 20 illustrates an expression mechanism 2014 attached to the bra 202 with the collection system 112 removed for clarity. The expression mechanism 2014 acts upon the breast with multiple helical members 2002 radiating outward along the breast in a spoke-like fashion from the nipple area. The helical members 2002 conform to the shape of the breast so that they contact the breast at multiple areas along their helixes. The helical members 2002 are supported and held to the bra 202 by driving supports 2004 and passive supports 2006 that are attached to bra 202 in a manner that allows a part of them to rotate freely along the longitudinal axis of helical members 2002 while the other part remains fixed to bra 202. The driving supports 2004 are connected to each other by a transmission ring 2008 that allows a single rotary input 2010 (FIG. 20a) from a motor 2012 (FIG. 20a) to rotate each of the driving supports 2004 and thus the helical members 2002. In the embodiment illustrated, a rack and multiple pinions are used to create the rotation of the helical members 2002. However, any other system can be used to rotate the helical members 2002 to fulfill the functions described herein.

The rotating motion of the helical members 2002 causes the contact areas between helical members 2002 and the breast to move toward the nipple creating an action that will cause or aid in the expression of milk from the nipple. Alternatively, the driving supports 2004 with transmission ring 2008 can be on the outer circumference of the device and the passive supports 2006 of the helical members 2002 can be at the inner ring of the mechanism 2014, in the proximity of the nipple. The user is able to control frequency of oscillation of the helical members 2002 with the controller of FIG. 1 to maximize the operating efficiency of the system.

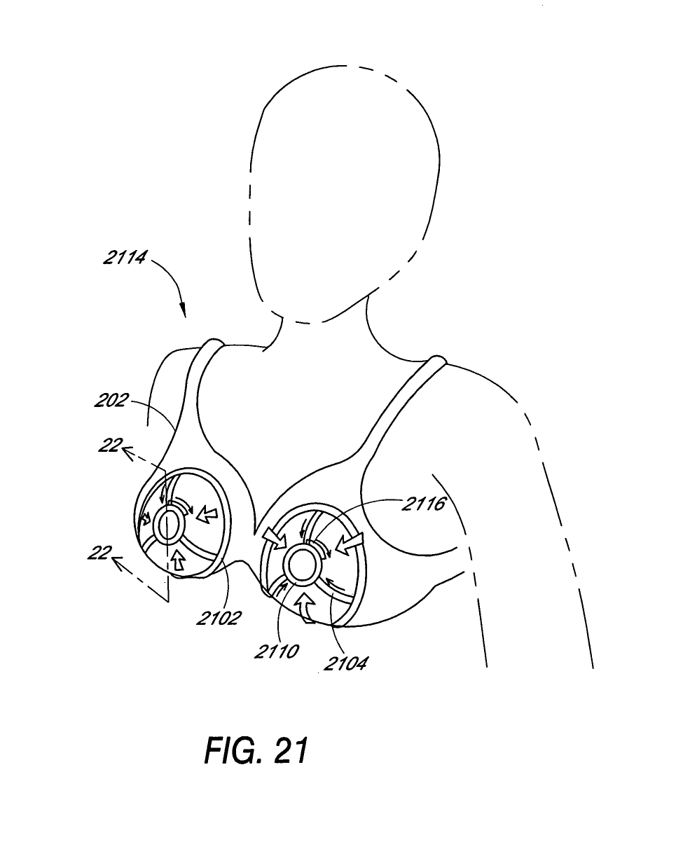

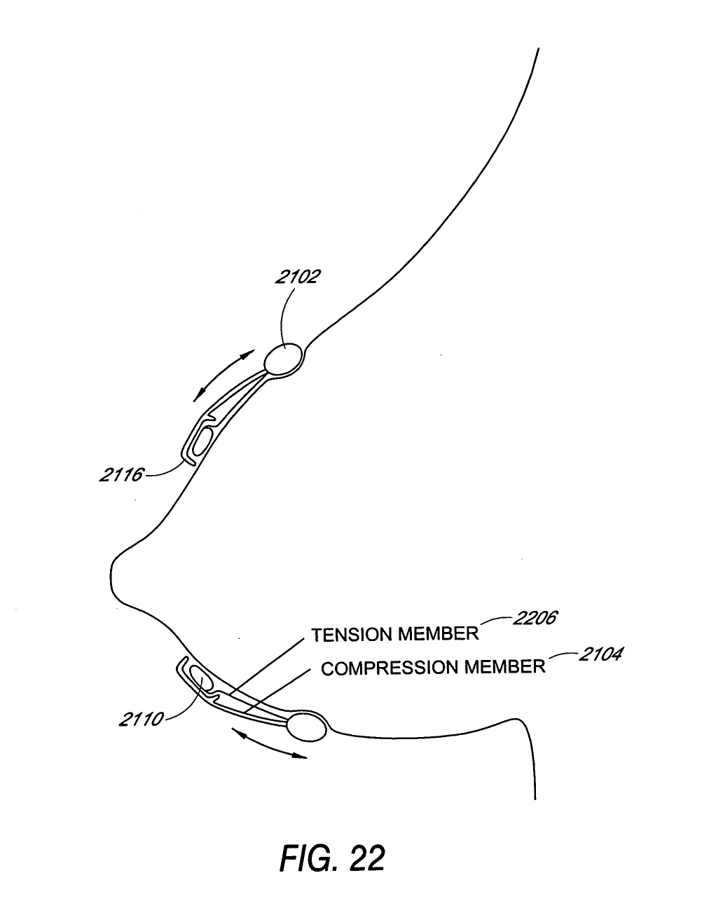

FIG. 21 illustrates another embodiment of an expression mechanism 2114 attached to the bra 202 with the collection system 112 removed for clarity. A ring 2102 around the breast is caused to contract towards the nipple. The ring 2102 is under tension in its expanded state and kept in the expanded state by radial compression stays or ribs 2104. FIG. 22 illustrates that the ring 2102 is caused to contract by radial tensioning members 2206 acting against the radial compression stays 2104, allowing contraction of the ring 2102. Release of the radial tension members 2206 causes the radial compression stays 2104 to force the ring to its expanded state.

The ring 2102 can be made of a rubbery or elastomeric material. Alternately, the ring can be made with rigid members capable of sliding over one another. The radial tension and compression members cause the ring 2102 to contract and expand, thereby massaging the breast. In one embodiment, the ring 2102 is caused to contract by rotation of an inner ring 2106. The tension members 2106 are connected at one end to the ring 2102 and then extend radially toward the nipple, where they are led through a guide 2116 then attached to the inner ring 2110. When the inner ring 2110 rotates, the end of the tension member 2106 that attaches to the inner ring 2110 moves away from the guide 2116, thereby pulling the end of the tension member 2106 attached to the ring 2102 toward the guide 2116 and collapsing the ring 2102. The rotation of the inner ring 2110 can be caused by a motor secured to the bra 202 or any other mechanism capable of rotating the inner ring 2110. Any other system for applying tension to the tension members 2106 can be used, as well.

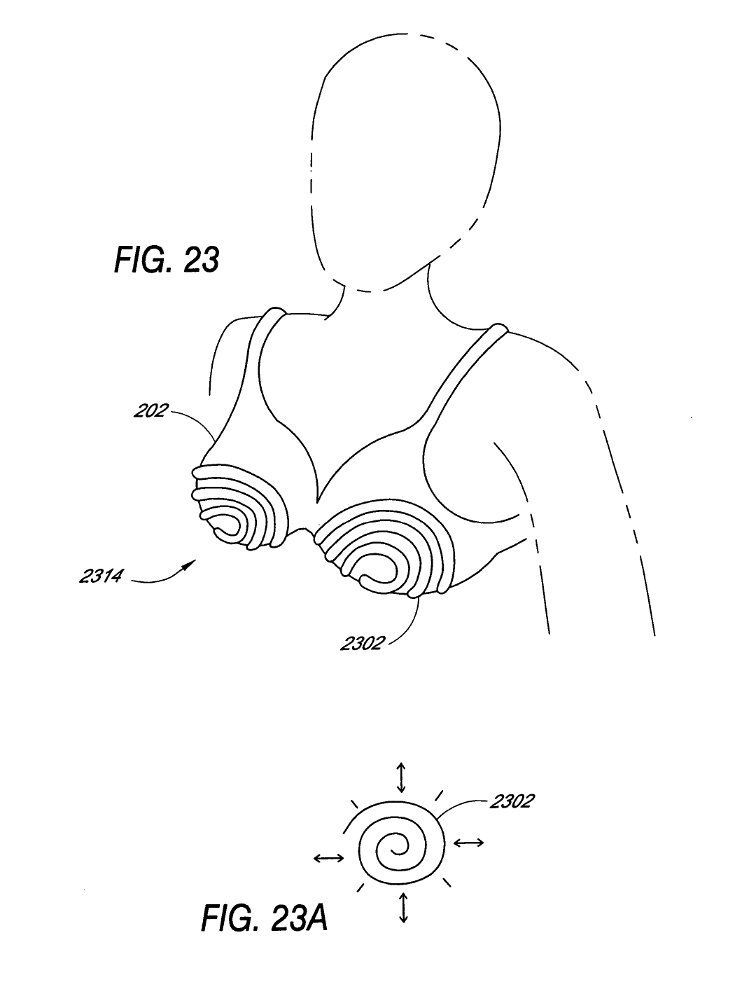

FIGS. 23 and 23a illustrate another embodiment of an expression mechanism 2314 attached to the bra 202 with the collection system 112 removed for clarity. The expression mechanism 2314 acts upon the breast through a spiral member 2302 formed in the bra 202. The ends of the spiral member 2302 are constrained by the bra 202. The spiral member 2302 is configured to expand and contract in length, causing the spiral to expand and contract radially. A motor (not shown), at the center or the outside edge, can induce length changes of the spiral member 2302 by applying and releasing tension from the spiral member 2302, causing the spiral member 2302 to coil and uncoil, thereby massaging the breast. However, any other mechanism for changing the length of the spiral member 2302 between the first end and the second end can be used.

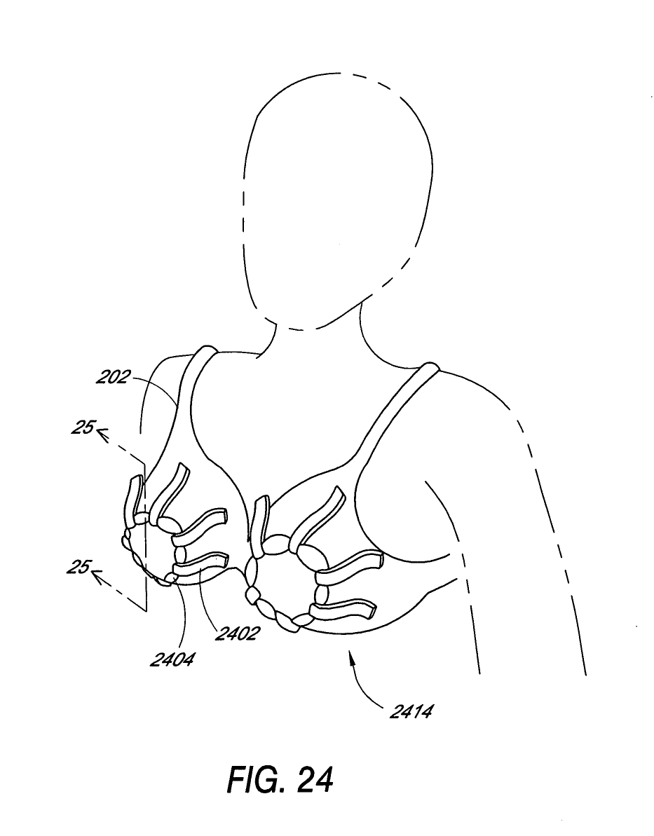

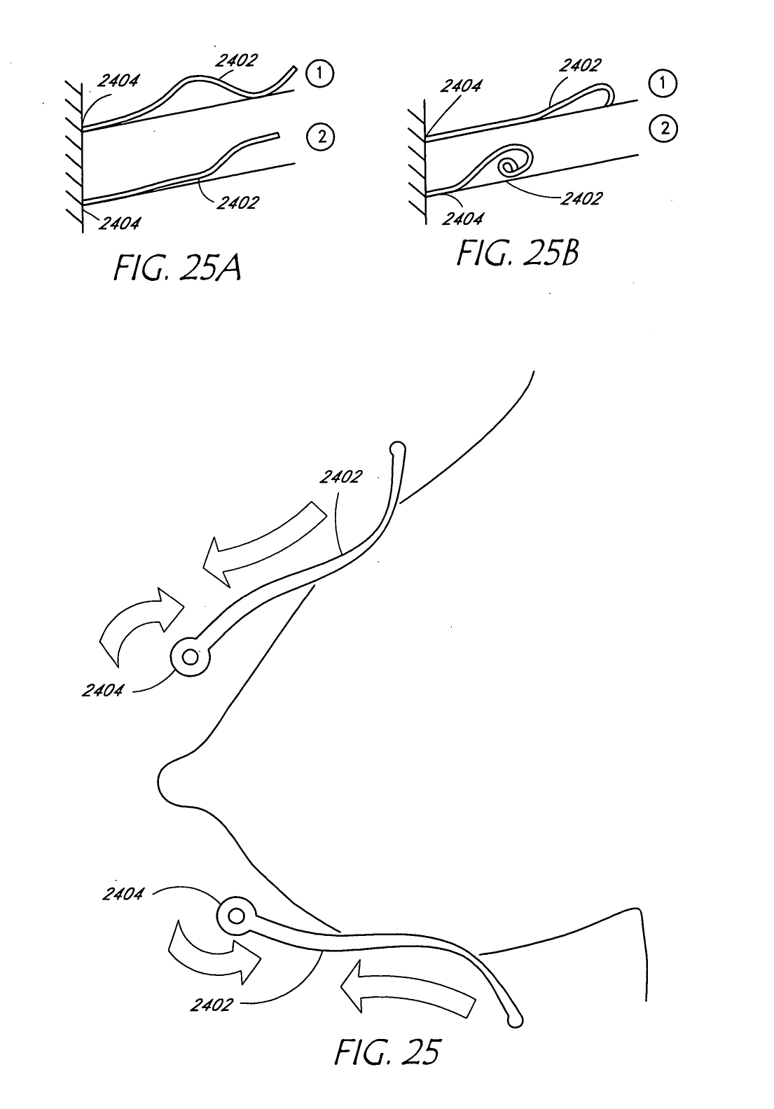

FIGS. 24 and 25 illustrate an expression mechanism 2414 attached to the bra 202 with the collection system 112 removed for clarity. The expression mechanism 2414 acts upon the breast with multiple flexible fingers 2402 radiating outward along the breast in a spoke-like fashion from the nipple area. The flexible fingers 2402 conform to the shape of the breast so that they contact the breast or bra 202 at an area along their length. The flexible fingers 2402 are connected to each other by a transmission ring 2404 that allows a single rotary input from an electromagnetic or pneumatic actuator (not shown) to rotate each of the flexible fingers 2402. Each of the flexible fingers 2402 can have one or more joints (not separately identified) located longitudinally along their lengths. The joints cause the flexible fingers 2402 to curl up when rotated. The joints can be rotated by pulling a tension member (not shown) in a manner similar to a tendon in a finger that passes through the joint and attaches to the finger 2402 near its outermost edge. A tension spring (not shown) centered at the joint, or the elasticity of the finger itself, if no joint is used, can return the flexible finger 2402 to an outward or straightened position when the pulling force of the tension member is removed. In this manner, the flexible fingers 2402 contact the breast first at an end 2406 furthest from the nipple. As the joints rotate, the flexible fingers 2402 curl so that the contact areas between flexible fingers 2402 and the breast move toward the nipple creating an action that will cause or aid in the expression of milk from the nipple.

In another embodiment illustrated in FIGS. 25, 25a and 25b, the flexible fingers 2502 can be made from metal that switches between alternated metallurgic forms when heat or electrical energy is applied to the metal. For example, one such shape-memory metal that can be used is Nitinol. An electrical current is applied to the flexible fingers 2502 causing the fingers to flex. FIG. 25a illustrates that massaging action can be achieved by varying the electrical current applied to the flexible fingers 2502. For example, when current is removed from the flexible fingers 2502 as in state 1, they can be flat and extended, and then when current is applied, as in state 2, they can begin to contract thereby moving the contact point near the nipple. However, any other variation of application of current or heat can be used as well.

Alternately, the flexible fingers 2502 can be pneumatically actuated. As the flexible fingers 2502 are pressurized and depressurized, they change shape and provide a massaging inward force. For example, as shown in FIG. 25b, the flexible fingers 2502 can unroll as they are pressurized as in state 1, then curl back up when the pressure is removed as in state 2. The contact point of the flexible finger 2502 exerting a force on the breast moves in a pattern from the point that extends away from a base of the flexible finger 2502 towards a point near the transmission ring 2404.

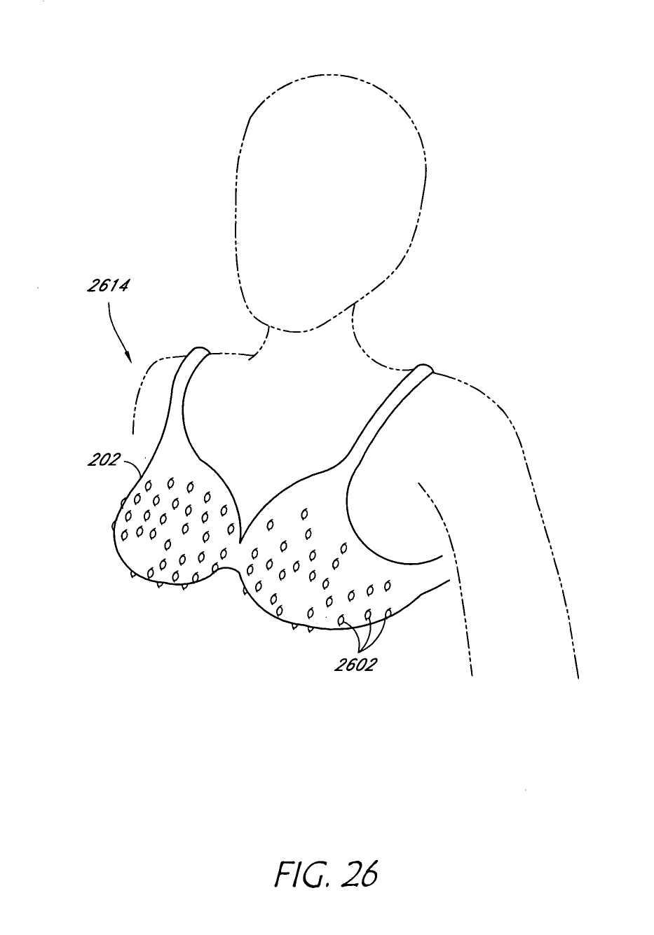

FIG. 26 illustrates another expression mechanism 2614 attached to the bra 202 with the collection system 112 removed for clarity. The expression mechanism 2614 acts upon the breast with multiple discrete pressure points 2602. The pressure points 2602 can made from a piezoelectric material or a memory metal such a nitinol, both of which can be actuated by a current sent through a wiring system (not shown) to the pressure point 2602. Additionally, in other embodiments ultrasonic, sonic vibrations or magnetism can be used for the pressure points 2602 with conductors directing the actuation current to the transducers at the pressure points 2602. The pressure points 2602 press against an area of the breast to stimulate the breast. When pressure is relieved, the pressure points 2602 no longer press on the breast. The pressure points 2602 can be arranged concentrically around the nipple (or in other radiating patterns) and are cyclically pressurized, or otherwise actuated, in order to produce patterns of pressure against the breast to assist in expressing milk. The user controls the pattern, frequency and pressure exerted by the pressure points 2602 with the controller 102 (FIG. 1) in order to maximize the effectiveness for milk expression. Some embodiments will have individual connections between each pressure point 2602 and the controller 102, while other embodiments will utilize banks of pressure points connected via a common connection with the controller 102.

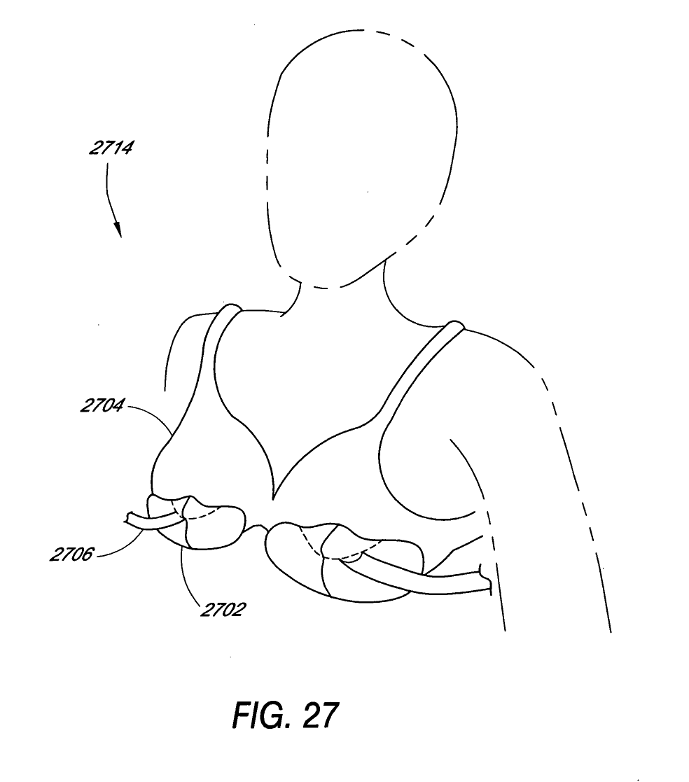

FIG. 27 illustrates another embodiment of an expression mechanism 2714. A removable device 2702 including any of the expression mechanisms and collection containers described above (not shown) can be used with a conventional bra 2704. The device 2702 is first inserted into a user's conventional bra 2704 and positioned according to user comfort. The device 2702 can be used to stimulate one area of the breast at a time. As desired, the woman can reposition the device 2702 to stimulate other areas of the breast. The milk is expressed via a duct, or collection tube 2706 as explained above. This collection tube 2706 then collects the milk in a collection container (not shown) positioned nearby. This embodiment is contemplated as useful with any of the expression mechanism embodiments described above.

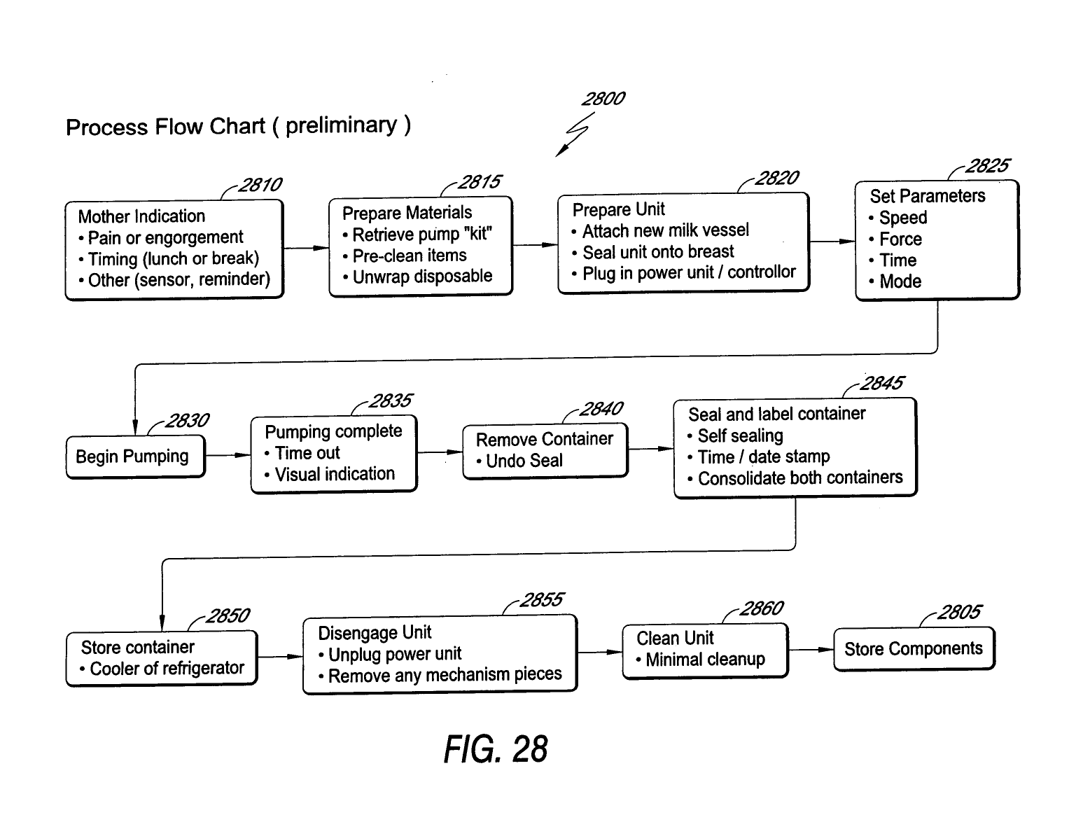

A function of some embodiments of the invention is achieved by a simple process 2800 illustrated in FIG. 28. At step 2810, the nursing mother determines that it is necessary to pump breast milk. At step 2815, the mother prepares the materials necessary to operate the breast pump system. At step 2820 the mother prepares the breast pump system by attaching a new milk collection container, placing a breast shield on and sealing the shield to one or both of her breasts and preparing the power source. At step 2825, the mother sets the user input parameters. At step 2830, the pump is actuated and the milk flows through the ducting to the collection container. At step 2835, pumping is completed either after the mother determines that sufficient milk has been pumped or the controller times out. At step 2840, the mother removes the collection container. At step 2845, the mother seals and labels the collection container. At step 2850, the container is stored until needed. At step 2855, the mother removes any mechanism pieces. At step 2860, the breast pump system is cleaned. At step 2865, the breast pump system is stored until the next time the mother desires to pump.

The invention has numerous attributes. The invention is a timesaving device for nursing mothers. Its strap-on design leaves the mother's hands free for tending to children or doing other things. The dual breast shields and expression mechanism express milk from both breasts at once, unlike other pumps that come with only one and take twice as long to perform the same function. No precious milk is wasted because mothers can express milk on a regular schedule. Babies will still get the benefits of breast milk, and mothers can store the milk for feedings when they cannot be home.

Specific parts, shapes, materials, functions and modules have been set forth, herein. However, a skilled technologist will realize that there are many ways to fabricate the system of the present invention, and that there are many parts, components, modules or functions that may be substituted for those listed above. While the above detailed description has shown, described, and pointed out the fundamental novel features of the invention as applied to various embodiments, it will be understood that various omissions and substitutions and changes in the form and details of the components illustrated can be made by those skilled in the art, without departing from the spirit or essential characteristics of the invention.

1. (canceled)

2. (canceled)

3. (canceled)

4. (canceled)

5. (canceled)

6. (canceled)

7. A breast milk expression system comprising:
an expression mechanism comprising a plurality of pressure members configured to apply pressure to a breast so as to express milk therefrom;
a flexible breast shield configured to be placed over the breast;
a conduit attaching to the breast shield and adapted to direct the flow of expressed milk from the breast; and
a collection container fluidly connected to the conduit and adapted to collect the expressed milk.

8. The system of claim 7, wherein each of the plurality of pressure members comprises a plurality of beads positioned along a length of a flexible member.

9. The system of claim 7, wherein each of the plurality of pressure members comprises a plurality of rollers positioned along a length of a flexible member.

10. The system of claim 7, wherein the plurality of pressure members comprises a plurality of helical members.

11. The system of claim 10, further comprising a motor configured to rotate the helical members.

12. The system of claim 7, wherein at least one of the plurality of pressure members is configured to apply pressure to the breast in response to an electrical current.

13. The system of claim 12, wherein the at least one of the plurality of pressure members comprises a memory metal.

14. The system of claim 12, wherein the at least one of the plurality of pressure members comprises a piezoelectric material.

15. The system of claim 7, wherein the plurality of pressure members comprises a plurality of flexible members and wherein the flexible members are attached to an actuator adapted to cause the flexible members to flex, thereby stimulating the breast.

16. The system of claim 7, wherein the at least one pressure member is an elongated member positioned adjacent to the breast and extending in a spiral configuration.

17. The system of claim 7, wherein the at least one pressure member comprises a plurality of pegs positioned along a length of a flexible member.

18. The system of claim 17, further comprising a motor configured to rock the pegs.

19. The system of claim 7, further comprising a bra configured to secure the collection container to a human torso and configured to secure each of the expression mechanism, the breast shield, and the conduit in a functional position adjacent to the breast.

20. A breast milk expression system comprising:
an expression mechanism comprising at least one pressure member configured to apply pressure to a breast so as to express milk therefrom;
a flexible breast shield configured to be placed over the breast;
a conduit attaching to the breast shield and adapted to direct the flow of expressed milk from the breast;
a collection container fluidly connected to the conduit and adapted to collect the expressed milk; and
a bra configured to secure the collection container to a human torso and configured to secure each of the expression mechanism, the breast shield, and the conduit in a functional position adjacent to the breast.

21. The system of claim 20, further comprising a motor configured to rotate the at least one pressure member.

22. The system of claim 20, wherein the at least one pressure member comprises a plurality of beads positioned along a length of a flexible member.

23. The system of claim 20, wherein the at least one pressure member comprises a plurality of rollers positioned along a length of a flexible member.

24. The system of claim 20, wherein the at least one pressure member comprises a plurality of pegs positioned along a length of a flexible member.

25. The system of claim 24, further comprising a motor configured to rock the pegs.

26. The system of claim 20, wherein the at least one pressure member comprises a plurality of helical members.

27. The system of claim 26, further comprising a motor configured to rotate the helical members.

28. The system of claim 20, wherein the at least one pressure member comprises a plurality of flexible members and wherein the flexible members are attached to an actuator adapted to cause the flexible members to flex, thereby stimulating the breast.

29. The system of claim 20, wherein the at least one pressure member is an elongated member positioned adjacent to the breast and extending in a spiral configuration.

30. The system of claim 29, further comprising a motor configured to rotate the at least one member.

31. The system of claim 20, wherein the at least one pressure members is configured to apply pressure to the breast in response to an electrical current.

32. The system of claim 32, wherein the at least one pressure member comprises a memory metal.

33. The system of claim 32, wherein the at least one pressure member comprises a piezoelectric material.
